(19) United States
(12) Patent Application Publication
(10) Pub. No.: US 2007/0012318 A1
(43) Pub. Date: Jan. 18, 2007

(54) Emergency medical restraint (EMR)

(75) Inventor: Stacy Howell Goodbread, Ohando, FL (US)

Correspondence Address: Stacy Howell Goodbread, 6000 Mattox ST., Ohando, FL, 32822, US

(73) Assignee: Stacy H. Goodbread

(21) Appl. No.: 11/474,230

(22) Filed: Jun. 26, 2006

Related U.S. Application Data
(60) Provisional application No. 60/698,379, filed on Jul. 12, 2005.

(51) Int. Cl. A61F 5/37; A61F 13/00

(52) U.S. Cl. 128/846; 128/870

(57) ABSTRACT

A new medical restraint devise designed to be used to secure, the arms, hands, and wrist of patients that have lost control of their upper extremities due to a medical or traumatic incident. This new medical devise in its design and constructed, positions the patients upper extremities in a neutral, centerline position of the patients body, removing the upper extremities from possible entanglements, providing the healthcare worker with the safe, unrestricted movement of the patient, provides full access to treatment sites and provides safe and secure methods of transportation of the patient with little or no interference.

### BACKGROUND OF INVENTION

The Emergency Medical Restraint (EMR) is used to properly and safely secure the unconscious, unresponsive, or cardiac arrest type patient's arms, hands, and wrist during the treatment, movement and transportation of medical or trauma patients. This invention was designed to solve the ongoing problems past and present of patient care issues that the healthcare worker faces in the treatment, movement and transportation of the unconscious, unresponsive, cardiac arrest type medical or trauma patients.

Due to the emergency condition of these types of patients, the patients have no control over there extremities, they become flaccid, extending beyond backboards and other transportation devices therefore interfering with the movement, treatment and the transportation of these patients.

The flaccid extremities of these patients during movement and transportation become entangled, or wedged in such objects but not limited to, furniture, doors, doorways, personnel, medical equipment, and emergency transport apparatus, entanglements also cause accidental damage, and disconnecting of emergency treatment such as IV,s monitors, airway adjuncts, and other advanced medical adjunctive devices.

These interferences also create hazardous working environments such as blood borne pathogen issues, traumatic injuries to the patient, bystanders and healthcare workers. In the past and at the present, healthcare workers continue in the unsuccessful attempt to overcome these interferences that cause unsafe and hazardous working environments by using unsecured and unsafe methods such as but not limiting to the placing of the patient's hands into clothing, between medical equipment, or using medical tape which causes injury to the patient as well as occludes circulation in the patients extremities. The Emergency Medical Restraint (EMR) address these important life safety issues and promotes safe and effective practices of securing the patient's extremities during the movement, treatment and transportation of the unconscious, unresponsive, or cardiac arrest type patients.

With the application of the Emergency Medical Restraint (EMR), the patient's extremities are drawn to a natural position within the center of the patient's body, safely exposing and securing vital areas for emergency treatment. With the extremities aligned in this neutral position, safe, proper and effective movement, treatment and transportation can be accomplished with minimal interference.

### SUMMARY OF THE INVENTION

The purpose and design of the Emergency Medical Restraint (EMR) is to assist the healthcare worker in the treatment, movement and transportation of the unconscious, unresponsive, cardiac arrest type medical or trauma patients, where as, the patient has no control over their extremities causing them to become flaccid, interfering with the treatment, movement and transportation of the patient.

The medical devise system will consist of hook and loop material, in various widths and lengths according to various patient sizes.

The medical devise consist of a one-piece T-shaped design construction, consisting of a wrist section which has two wrist straps and a tension section, consisting of the tension strap. The two wrist straps located in the wrist section of the medical devise are to be secured around the patient's left and right wrist, passed through a d-ring buckle system and fastened by means of a hoop and loop fastening system. The tension strap located in the tension section of the medical devise is designed to be attached to patient transporting devices and secured with a hook and loop fastening system. The tension strap in the tension section of the medical devise can be adjusted as needed according to various patient sizes, as the tension strap is adjusted; the hands, wrist and arms of the flaccid unconscious, unresponsive, cardiac arrest type patient are drawn into a neutral position towards the center of the patient's body.

With the hands, wrist, and arms drawn into this neutral position, key treatment sites are exposed, giving the healthcare worker proper treatment access to the patient with minimal interference, also providing the means of safe, efficient movement of the patient, which in turn, creates a safe non-hazardous working environment.

### BRIEF DESCRIPTION OF THE DRAWINGS

FIG. 1 is the wrist section of the present invention, constructed of hook and loop material containing the two wrist straps, with a d-ring buckling system for each wrist strap, using hook and loop fastening system to secure the wrist strap onto itself.

FIG. 2 is the tension section of the present invention, constructed of hook and loop material, this section contains the tension strap; using hook and loop fastening systems to secure the tension strap onto itself.

FIG. 3 is a view of entire one-piece devise of the present invention which includes FIG. 1 and FIG. 2. This view demonstrates the present invention secured to a transportation devise.

### DETAILED DESCRIPTIONS OF THE PREFERRED INVENTION

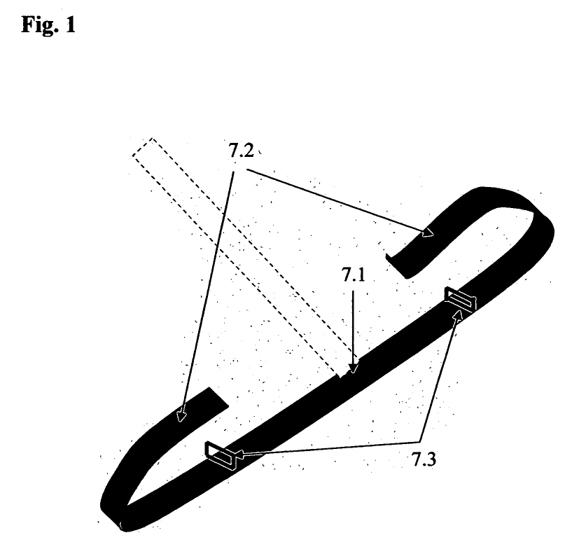

FIG. 1 is the view of the wrist section of the present invention. 7.1 depicts of the location where the tension section meets the wrist section and is sown together. 7.2 depict the wrist straps which secure the left and right wrist of the patient. 7.3 depicts the d-ring buckling system in which the wrist straps 7.2 inserts through and tightens over the patients left and right wrist, the wrist straps 7.2 may vary in length depending on the patient size. Once the wrist strap 7.2 is tightened to desire tightness, it will be secured onto itself with a hook and loop fastener system.

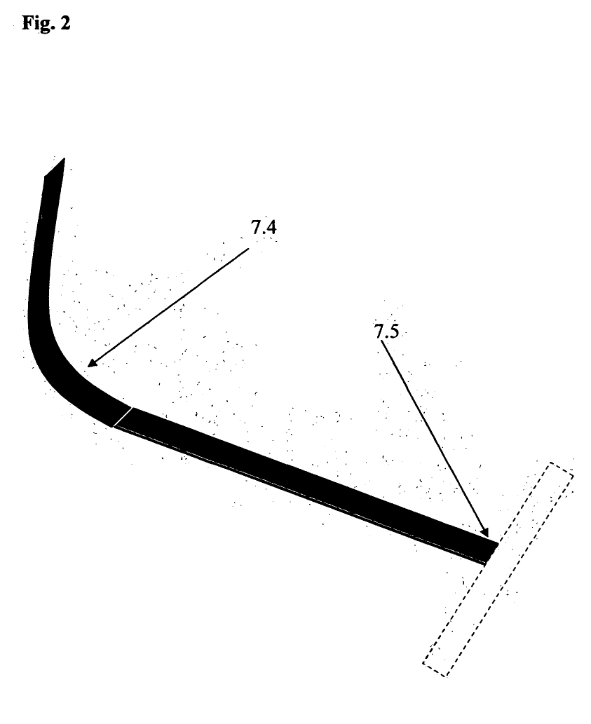

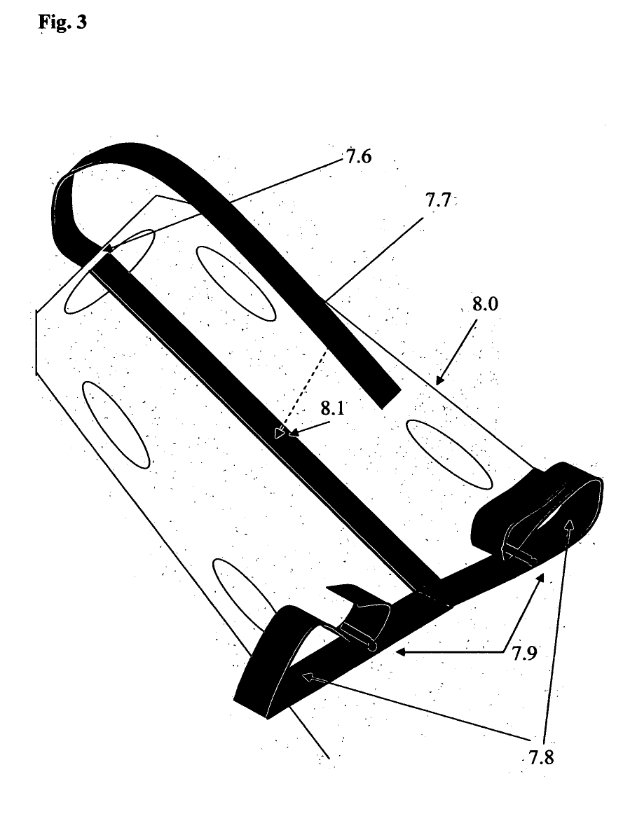

FIG. 2 is a view of the tension section of the present invention. 7.4 depict the area of the tension strap that would secure around the transportation devises, once the tension strap has been adjusted to the proper tension, depending upon patients height, the tension strap will then be secured onto itself with a hook and loop fastener system. 7.5 depict the area on the tension section that would meet and connect to the wrist section in FIG. 1, 7.1.
FIG. 3 views the entire one-piece devise of the present invention in it's relation to being secured around the transportation devise. 7.6 depicts the tension strap of the tension section being secured around transportation devise and 7.7 depicting the area of the tension strap of the tension section that is used to adjust the amount of tension needed to achieve the desired neutral position of the patient's hands, arms and wrist, depending upon the height and size of the patient. Once the desired position is achieved, 7.7 of the tension strap would then be secured with a hook and loop fastener system onto the tension section of itself. 8.1. 7.8 depicts the wrist straps that secure the patient's left and right wrist, the patient's wrist are secured by feeding the wrist straps through the d-ring buckling system 7.9, and tightened as needed to reach the desired effect, depending upon the size of the patient's wrists. Once desired effect is achieved the wrist straps 7.8 is then secured onto itself by a hook and loop fastener system. 8.0 depict the transportation device on which the patient is placed and transported.

During medical or traumatic emergencies where patients are in a mental state were they lose all control of their extremities, such as but not limited to the unconscious, unresponsive, cardiac arrest type patients, treatment, movement, and transportation of these patient can be very difficult, dangerous and can create health hazards for the patients, and healthcare workers, leaving an unsafe working environment. The present invention is designed to safely secure these types of patients, by securing the extremities such as hands, arms, and wrist to a safe neutral position, a safe, and hazard free working environment will be created, given the healthcare worker the ability to treat, secure, move, and transport the patient with no interferences.

What I claim is:

1. A patient treatment, movement and transportation restraint device one-piece T-shaped in design, consisting of two sections, the wrist section and the tension section. The said wrist section portion of the invention consist of two wrist straps for left and right wrist, two d-ring buckling system located on the wrist section with one of the d-ring buckling system for the left wrist strap and one of the d-ring buckling system for the right wrist strap. The wrist straps are designed with a hook and loop fastening system that enables the wrist strap to be fastened onto itself. The said tension section portion of the present invention consist of a long tension strap, the tension strap is designed to create tension onto the arms, hands and wrist section of the said invention. Tension is accomplished by attaching the said tension strap around a portion of a transportation device, pulling tension onto the said tension strap which in turn causes tension to be placed onto the said wrist section causing the upper exterminates to be drawn inward towards to centerline of the patients body, eliminating the possibility of the entaglement of the upper extremities.

2. The present invention according to claim 1, wherein:
Said invention is designed to safely secure and restraint the hands, arms, and wrist of medical and traumatic patients that have no control over their extremities.

3. The present invention according to claim 1, wherein:
Said present invention is designed to be used in conjunction with any transportation devise.

4. The present invention according to claim 1, wherein:
Said present invention is designed to bring flaccid uncontrollable extremities towards the centerline of the patient's body.

5. The present invention according to claim 1, wherein:
Said present invention is designed to eliminate the unsafe and hazardous working environment, and create a safe effective method of securing, exposing vital treatment sites, the treatment, movement, and transportation of patients that has lost all control of their extremities.

6. The present invention according to claim 1, wherein:
The wrist section portion of the said invention consist of two wrist straps designed to secure and restrain the patient's upper extremities, hands, arms, and wrist.
The said wrist section portion is constructed of hook and loop material, width, and length of said invention can vary depending on the size and age of the patient.

7. The present invention according to claim 1, wherein:
The wrist section portion of the said invention consist of a d-ring buckling system, this said d-ring buckling system may vary in size depending upon the width of wrist strap.

8. The present invention according to claim 1, wherein:
Said present invention consist of a hook and loop fastening system that allows the wrist strap to be fastened onto itself, the hook and loop fastening system design to adhere to itself by means of hook and loop fastening material.

9. The present invention according to claim 1, wherein:
The wrist strap is designed to secure around the patients wrist, using the d-ring buckling system, the wrist strap is passed through the d-ring buckling system, and tightened as needed according to the size of the patient's wrist, securing the patients wrist. The wrist straps hook and loop fastening system is designed to be fastened onto itself once required tension is achieved on the wrist strap and patient's wrist causing the patient's hands, arms, and wrist to be drawn to a neutral and safe position, centerline of the patient.
